(19) United States
(12) Patent Application Publication
(10) Pub. No.: US 2007/0292448 A1
(43) Pub. Date: Dec. 20, 2007

(54) Preloaded dendritic cell vaccines for treating cancer

(76) Inventors: Jane S. Lebkowski, Portola Valley, CA (US); Anish Sen Majumdar, Cupertino, CA (US); William D. Stempel, Palo Alto, CA (US); J. Michael Schiff Schiff, Menlo Park, CA (US)

Correspondence Address: GERON CORPORATION, 230 CONSTITUTION DRIVE, MENLO PARK, CA, 94025, US

(21) Appl. No.: 11/710,244

(22) Filed: Feb. 23, 2007

Related U.S. Application Data
(63) Continuation of application No. 11/202,319, filed on Aug. 10, 2005, now abandoned.
(60) Provisional application No. 60/600,639, filed on Aug. 10, 2004.

(51) Int. Cl. A61K 39/00; A61P 43/00; C12N 5/06

(52) U.S. Cl. 424/185.1; 435/372

(57) ABSTRACT

This disclosure provides a technology for making a dendritic cell vaccine suitable for high volume manufacturing and distribution. Human stem cells are differentiated in a multi-step protocol to generate cell populations bearing a dendritic cell phenotype. The cells are loaded by pulsing with a specific tumor antigen, or by activation of an inducible transgene. The primed dendritic cells are powerful components of a vaccination strategy to elicit an immune response against tumor-associated antigens like telomerase. Vaccines and reagent combinations prepared according to this invention can be used on demand as off-the-shelf products for treating cancer.

### RELATED APPLICATIONS

This application claims the priority benefit of U.S. Provisional Patent Application 60/600,639 (Docket 138/001x), filed Aug. 10, 2004.

The priority application is hereby incorporated herein in its entirety, with respect to dendritic cells containing tumor associated antigens such as telomerase reverse transcriptase (TERT), and their manufacture and use in vaccine formulations for the treatment of cancer.

### BACKGROUND

Biotechnology has brought a brave new era to the treatment of cancer with the development of monoclonal antibodies for specific cancer types. Herceptin® (Trastuzumab), Rituxan® (rituximab), and CamPath® (alemtuzumab) have been a clinical and commercial success. But these medicines provide only passive treatment without recruiting constructive participation by the host's immune system. They also leave out what may be the most powerful immune effector mechanism for causing tumor regression: the cytotoxic T lymphocyte (CTL) compartment.

Considerable effort is underway in laboratories all over the world to find an active vaccine that will overcome the natural tolerance to self-antigens, and induce a strong anti-tumor CTL response.

Peptide vaccines have been developed based on tumor associated antigens like carcinoembryonic antigen (CEA) or gp100, sometimes with epitope enhancement to enhance immunogenicity (S. A. Rosenberg et al., Nat. Med. 4:321, 1998). Cytokines, chemokines, or costimulatory molecules have been used as potential adjuvants (J. A Berzofsky et al., Nat. Rev. Immunol. 1:209, 2001; J. D. Ahlers et al., Proc. Nat. Acad. Sci. USA 99:13020, 2002). Active immune response to tumor antigen has also been achieved in cancer patients using anti-idiotype antibody, made to mimic the target antigen while providing further immunogenicity (U.S. Pat. Nos. 5,612,030 and 6,235,280). Nucleic acid vectors based on adenovirus, vaccinia, and avipox encoding such as CEA or prostate specific antigen (PSA) are also in clinical trials (J. L. Marshall et al., J. Clin. Oncol. 18:3964, 2000; M. Z. Zhu et al., Clin. Cancer Res. 6:24, 2000; I. M. Belyakov et al., Proc. Natl. Acad. Sci. USA 96:4512, 1999).

Tumor cell vaccines have also been based on tumor cells taken either from the patient being treated, or from an autologous source bearing a similar profile of tumor antigens. They are genetically modified to express a cytokine like GM-CSF or IL-4 that is thought to recruit the host immune system (J. W. Simons et al., Cancer Res. 59:5160, 1999; R. Soiffer et al., Proc. Natl. Acad. Sci. USA 95:13141, 1998; E. M. Jaffee et al., J. Clin. Oncol. 19:145, 2001; R. Salgia et al., J. Clin. Oncol. 21:624, 2003). Transfected tumor cell vaccines are in late-stage clinical trials for prostate cancer, lung cancer, pancreatic cancer, and leukemia (R. Salgia et al., J. Clin. Oncol. 21:624, 2003; K. M. Hege et al., Lung Cancer 41:S103, 2003).

An improved version of this approach is to isolate the patient's own tumor cells, and combine them with a cell line transfected to express a cytokine like GM-CSF in membranes form (U.S. Pat. No. 6,277,368). The transfected cells recruit the host immune system, which then initiates a strong CTL response against the tumor cells as bystanders. Another type of cellular vaccine comprises a patient's tumor cells combined with alloactivated T lymphocytes, which again play the role of recruiting the host immune system (U.S. Pat. Nos. 6,136,306; 6,203,787; and 6,207,147).

Because dendritic cells play a central role in presenting tumor antigen to prime the CTL compartment, there has been considerable research interest in autologous dendritic cells as a tumor vaccine (G. Schuler et al., Curr. Opin. Immunol. 15:138, 2003; J. A. Berzofsky et al., J. Clin. Invest. 113:1515, 2004). Clinical trials have been based on dendritic cells from two sources: a) purified DC precursors from peripheral blood (L. Fong & E. G. Engleman, Annu. Rev. Immunol. 15:138, 2003); and b) ex vivo differentiation of DCs from peripheral blood monocytes (F. Sallusto et al., J. Exp. Med. 179, 1109, 1994) or CD34+ hematopoietic progenitor cells (J. Banchereau et al., Cancer Res. 61:6451, 2001; A. Makensen et al., Int. J. Cancer 86:385, 2000).

U.S. Pat. Nos. 5,853,719 and 6,306,388 (Nair et al.) describe methods for treating cancers and pathogen infections using antigen-presenting cells loaded with RNA. U.S. Pat. Nos. 5,851,756, 5,994,126, and 6,475,483 (Rockefeller Univ., Merix Bioscience Inc.) disclose methods for in vitro proliferation of dendritic cell precursors and their use to produce immunogens. U.S. Pat. Nos. 6,080,409 and 6,121,044 (Dendreon) outline antigen presenting cell compositions and their use for immunostimulation.

D. Boczkowski et al. (J. Exp. Med. 184:465, 1996) reported that dendritic cells pulsed with RNA are potent antigen-presenting cells in vitro and in vivo. S. K. Nair et al. (Eur. J. Immunol. 27:589, 1997) reported that antigen-presenting cells pulsed with unfractionated tumor-derived peptides are potent tumor vaccines. F. O. Nestle et al. (Nat. Med. 4:328, 1998) reported vaccination of melanoma patients with peptide- or tumor lysate-pulsed dendritic cells. B. Thurner et al. (J. Exp. Med. 190:16169, 1999) reported vaccination with mage-3A1 peptide-pulsed dendritic cells in Stage IV melanoma. L. Fong et al. (J. Immunol. 167:7150, 2001) described dendritic cell-based xenoantigen vaccination for prostate cancer immunotherapy.

A. Heiser et al. (Cancer Res. 61:338, 2001; J. Immunol. 166:2953, 2001) reported that human dendritic cells transfected with renal tumor RNA stimulate polyclonal T cell responses against antigens expressed by primary and metastatic tumors. C. Milazzo et al. (Blood 101:977, 2002) reported the induction of myeloma-specific cytotoxic T cells using dendritic cells transfected with tumor-derived RNA. Z. Su et al., (Cancer Res. 63:2127, 2003) reported immunological and clinical responses in metastatic renal cancer patients vaccinated with tumor RNA-transfected dendritic cells.

The invention described here provides important advances in vaccine technology. It makes effective cellular vaccines more accessible and affordable for clinicians and cancer patients everywhere.

### SUMMARY

This disclosure provides new dendritic cell vaccines for eliciting an immune response against tumor targets, thereby contributing to treatment of the cancer.

Unlike previously available technology, the vaccines of this invention are designed as off-the-shelf products. The cellular component of each vaccine is made en masse from pluripotent progenitor cells. The antigen-presenting cells are distributed either preloaded or in combination with tumor antigen. With this technology in place, there is no need to harvest cells or tissue from the patient, and there is no need to process tissue into a vaccine in a patient-specific manner. Rather, the vaccine is used right out of the package, or after minimal processing—thereby allowing the patient to be treated as soon as appropriate, and at much lower cost.

The cellular compositions are made from stem cells, particularly pluripotent stem cells of human origin. The culture is differentiated into cells having characteristics of dendritic cells, loaded with a specific target antigen on the tumor cell, and formulated for administration to a human subject. The differentiation process involves culturing the cells in an environment of cytokines and other factors that generate a hematopoietic or early dendritic cell progenitor, and then maturing the cells to the phenotype intended for administration. Effective factor combinations and markers to effect and monitor the differentiation procedure are provided later in the disclosure.

An exemplary tumor antigen for loading into the cells is the catalytic component of the telomerase enzyme (TERT), which most tumors require for ongoing replication. Full-length human TERT can be used (optionally in an inactive form), or any fragment that contains an immunogenic epitope. Other tumor targets effective alone or in combination with TERT are listed in a later section.

The cells are loaded with tumor antigen in a manner that allows the cells to present the antigen to the host immune system at an appropriate time. The cells can be genetically modified with mRNA encoding tumor antigen near the time of administration, or pulsed with antigen in the form of a protein complex. Alternatively, the cells can be transduced at any stage in the differentiation pathway with a recombinant gene that causes tumor antigen to be expressed in the end-stage dendritic cell. As an option, tumor antigen can be expressed under control of an inducible promoter. This allows the kinetics of antigen pulsing to be mimicked by combining with the compound that induces the promoter near the time of administration, thereby initiating antigen presentation.

Dendritic cells loaded with tumor antigen can then be administered to a patient having a tumor in order to elicit an immune response (ideally cytotoxic CD8+ T lymphocytes with CD4+ help). The effect of early stage dendritic cells can be enhanced by treating the injection site with an adjuvant that promotes maturation, such as imiquimod or polyarginine (S. Nair et al., J. Immunol. 171:6275, 2003; WO 04/053095). If necessary, reactivity against the histocompatibility type of the cells can be decreased by pretreating the patient with toleragenic dendritic cells made from the same cell line. The antigen-loaded dendritic cells are then administered to the patient in a series that initiates an immunological or therapeutic response. Once initiated, the response can be maintained or boosted by further periodic administration of the loaded dendritic cells, or with the tumor antigen in another form (such as a peptide vaccine, or a viral or plasmid vector).

Embodiments of the invention include methods for differentiating the dendritic cells from pluripotent stem cells, methods of loading the cells with select tumor antigen, early or late stage dendritic cells obtainable by such methods, and the use of the cells for making medicaments, eliciting an immune response, or treating cancer. Other embodiments are product combinations for use in manufacture, testing, or clinical therapy: e.g., the dendritic cells of this invention in combination with the stem cell line from which they were derived, tumor antigen or mRNA, a maturation agent, an expression inducing compound, a second toleragenic cell population, or a tumor vaccine in a different formulation.

Other embodiments of the invention will be apparent from the description that follows.

### DRAWINGS

FIG. 1 is a differentiation paradigm for making dendritic cells from human pluripotent stem (hPS) cells. The cells are cultured with factors that direct or promote formation of precursors for the broad category of hematopoietic cells; which in turn are directed into a dendritic cell lineage using a second factor combination. Markers are shown for determining phenotype, although the cells need not have all the markers in order to have the desired properties. The cells can be loaded with tumor antigen as protein or mRNA just before administration or a final maturation step. Alternatively, they can transduced with a gene that causes antigen expression, either before differentiation or at an intermediate stage.

FIG. 2 is another differentiation paradigm in which hPS cells are directed towards phagocytic cells from the outset. Again, there is a plurality of different factor combinations used sequentially, but early intermediates already bear hallmarks of monocytic cells of the phagocytic or dendritic lineage. The cells can be pulsed with tumor antigen protein or mRNA when they have the properties of phagocytic cells, or transduced with an expression vector at any stage of differentiation.

FIG. 3 is an overview of a clinical trial in which autologous dendritic cells were generated from peripheral blood adherent cells, and loaded with mRNA for human TERT (in some cases also including a LAMP-1 lysosomal trafficking sequence). Patients with metastatic prostate cancer were administered with the vaccine for 3 or 6 weeks, and monitored for their response.

FIG. 4 shows the delayed-type hypersensitivity reactions observed at the injection site. Both CD8 and CD4 T lymphocytes were present beginning at vaccine cycle two, showing a rapid cellular immune response.

FIG. 5 shows cytokine expression profiles of vaccine-induced TERT-specific CD4+ T lymphocytes isolated from peripheral blood of treated patients. The expression profile is consistent with a Th-1 type antigen-specific cellular immune response.

FIG. 6 shows the kinetics of telomerase-specific cytotoxic T lymphocyte response as determined by ELISPOT assay. CD8+ antigen-specific cytotoxic T cells are present in the circulation as early as one week after the first vaccination. After the sixth injection, the level climbed to about 2% of the total circulating pool. This level is remarkable, because it equates to what is typically observed following administration of vaccines for foreign antigens such as PPD.

FIG. 7 shows the clinical status of patients who were treated. Most of the patients vaccinated six times had the rise in PSA levels stopped by the vaccine. The level of circulating tumor cells measured in these patients prior to immunization was 100- to 1000-fold higher than normal (horizontal line), but reverted to normal (undetectable) levels and remained there for 3 months after treatment.

### DETAILED DESCRIPTION

This disclosure provides a system for making and using cellular vaccines for treating cancer. Dendritic cells present tumor antigen to the host immune system in a manner that elicits an anti-tumor immune response, or otherwise improves the potential outcome of a patient having a tumor.

The invention is an advance over previous dendritic cell vaccines, because the composition may be prepared in advance as an off-the-shelf pharmaceutical product, suitable for administration for the treatment of cancer in a non-patient-specific manner.

Current dendritic cell vaccines are made from a patient's own peripheral blood mononuclear cells, which need to be collected and cultured in a manner that enriches for antigen presenting cells. Current whole tumor vaccines are made from a patient's own tumor tissue, which is extracted for tumor-specific antigen or mRNA for combining into the vaccine preparation. The cultured dendritic cells are then pulsed with the tumor cell extract to produce a patient-specific vaccine. In spite of the clinical success of this type of vaccine, there are substantial resource and financial investments required that are not available for all patients. Furthermore, there may be significant time delay in preparing the components of the vaccine for each patient, which may prevent them from being treated as soon as appropriate in their clinical care.

The new system described in this disclosure addresses these issues in the following way. First, the dendritic cells can be made not from the patient's own blood cells, but from a common stem cell line that is both self-renewing, and capable of generating enough antigen presenting cells for an off-the-shelf pharmaceutical. Second, the cells are primed not with whole tumor extract, but with one or more defined tumor antigens selected for their immunogenic properties and critical role in tumor progression. Third, activation of the cells can be done using a previously prepared tumor antigen preparation that can be combined with the cells just before administration, or by genetically engineering the cells to produce the antigen internally.

These features place the technology of highly powerful dendritic cell vaccine compositions into the hands of a clinician in general practice for the first time. Since the compositions and reagents of this invention are provided in prepackaged form, the clinician has the option of implementing the technology without elaborate and extensive extraction and tissue culturing facilities. Instead, the patient is administered immediately upon demand with the packaged pharmaceutical products obtained from a commercial supplier. The clinician can then turn her attention to the general management of the patient's condition, and monitor the patient's response to treatment.

Sources of Stem Cells

This invention can be practiced with stem cells of various types. Preferred are pluripotent cells that have both a broad differentiation capacity, and considerable replicative capacity.

Prototype “human Pluripotent Stem cells” (hPS cells) are pluripotent cells derived from pre-embryonic, embryonic, or fetal tissue at any time after fertilization, and have the characteristic of being capable under appropriate conditions of producing progeny of several different cell types that are derivatives of all of the three germinal layers (endoderm, mesoderm, and ectoderm), according to a standard art-accepted test, such as the ability to form a teratoma in 8-12 week old SCID mice. Unless otherwise specified, hPS cells are not derived from a cancer cell or other malignant source. It is desirable (but not always necessary) that the cells be euploid.

Exemplary are embryonic stem cells and embryonic germ cells used as existing cell lines or established from primary embryonic tissue of human origin. This invention can also be practiced using pluripotent cells obtained from primary embryonic tissue, without first establishing an undifferentiated cell line.

The skilled reader will appreciate that some aspects of this invention can be practiced using dendritic cells sourced from hematopoietic tissue and differentiated by established protocols. The information provided later in this disclosure for engineering the cells to express a tumor specific antigen (either through a standard expression vector, or using an inducible promoter) provides a substantial advance in dendritic cell vaccines made by previously established protocols. Suitable sources of hematopoietic cells are peripheral blood mononuclear cells separated from whole blood, adherent cells from a leukapheresis preparation, cells obtained from a bone marrow tap, and cord blood. General information on the sourcing and culturing of hematopoietic cells can be found in U.S. Pat. Nos. 4,714,680; 5,061,620; 5,460,964; 5,474,687; and 5,610,056; and in Bonde J et al., Curr Opin Hematol. 11:392, 2004; Nakano et al., Trends Immunol. 24:589, 2003; Conrad et al., J Leukoc. Biol. 64:147, 1998; and de Vries et al., Methods Mol. Med. 109:113, 2005.

The culturing and differentiation of stem cells is described generally in the current edition of Culture of Animal Cells: A Manual of Basic Technique (R. I. Freshney ed., Wiley & Sons); General Techniques of Cell Culture (M. A. Harrison & I. F. Rae, Cambridge Univ. Press), Embryonic Stem Cells: Methods and Protocols (K. Turksen ed., Humana Press), Differentiation of Embryonic Stem Cells (Methods in Enzymology, 365) by P. M. Wassarman & G. M. Keller, Academic Press, 2003; and Adult Stem Cells by K. Turksen, Humana Press, 2004.

Other publications on stem cell differentiation or use include the following: U.S. Pat. No. 6,280,718 (Kaufman & Thomson); and U.S. Pat. No. 6,368,636 (Osiris); WO 02/44343 (Geron); WO 03/050251 (Robarts Inst.); WO 98/06826 (Baxter); WO 03/083089 (Moore, PPL Therapeutics); WO 00/28000 (Fairchild et al.); WO 02/072799 (G. Schuler et al.); D. S. Kaufman et al., J. Anat. 200(Pt. 3):243, 2002; F. Li, J. A. Thomson et al., Blood 98:335, 2001; G. R. Honig, F. Li et al., Blood Cells Molec. Dis. 32:5, 2004; T. Schroeder et al., Br. J. Haematol. 111:890, 2000; P. J. Fairchild et al., Transplantation 76:606, 2003; P. J. Fairchild et al., Curr. Biol. 10:1515, 2000; S. T. Fraser et al., Meth. Enzymol. 365:59, 2003; H. Matsuyoshi et al., J. Immunol. 172:776, 2004; B. Obermaier et al., Bio. Proced. Online 5:197, 2003; S. Senju et al., Blood 101:3501, 2003; K. Moore et al., Arterioscler. Thromb. Vasc. Biol. 18:1647, 1998; M. Mohamadzadeh et al., J. Immune Based Ther. Vaccines 2:1, 2004; Fairchild et al., Int. Immunopharmacol. 5:13, 2005; and Zhan et al., Lancet 364:163, 2004.

Embryonic Stem Cells

Embryonic stem cells can be isolated from blastocysts of primate species (U.S. Pat. No. 5,843,780; Thomson et al., Proc. Natl. Acad. Sci. USA 92:7844, 1995). Human embryonic stem (hES) cells can be prepared from human blastocyst cells using the techniques described by Thomson et al. (U.S. Pat. No. 6,200,806; Science 282:1145, 1998; Curr. Top. Dev. Biol. 38:133, 1998) and Reubinoff et al, Nature Biotech. 18:399, 2000. Equivalent cell types to hES cells include their pluripotent derivatives, such as primitive ectoderm-like (EPL) cells, outlined in WO 01/51610 (Bresagen).

hES cells can be obtained from human preimplantation embryos (Thomson et al., Science 282:1145, 1998). Alternatively, in vitro fertilized (IVF) embryos can be used, or one-cell human embryos can be expanded to the blastocyst stage (Bongso et al., Hum Reprod 4:706, 1989). The zona pellucida of the blastocyst is removed, and the inner cell masses are isolated. The intact inner cell mass can be plated on mEF feeder layers, and after 9 to 15 days, inner cell mass derived outgrowths are dissociated into clumps, and replated. ES-like morphology is characterized as compact colonies with apparently high nucleus to cytoplasm ratio and prominent nucleoli. Resulting ES cells are then routinely split every 1-2 weeks.

hPS cells can be propagated continuously in culture, using culture conditions that promote proliferation while inhibiting differentiation. Traditionally, ES cells are cultured on a layer of feeder cells, typically fibroblasts derived from embryonic or fetal tissue (Thomson et al., Science 282:1145, 1998).

Scientists at Geron have discovered that hPS cells can be maintained in, an undifferentiated state even without feeder cells. The environment for feeder-free cultures includes a suitable culture substrate, such as Matrigel® or laminin. The cultures are supported by a nutrient medium containing factors that promote proliferation of the cells without differentiation (WO 99/20741). Such factors may be introduced into the medium by culturing the medium with cells secreting such factors, such as irradiated primary mouse embryonic fibroblasts, telomerized mouse fibroblasts, or fibroblast-like cells derived from hPS cells (U.S. Pat. No. 6,642,048). Medium can be conditioned by plating the feeders in a serum free medium such as Knock-Out DMEM (Gibco), supplemented with 20% serum replacement (US 2002/0076747 A1, Life Technologies Inc.) and 4 ng/mL bFGF. Medium that has been conditioned for 1-2 days is supplemented with further bFGF, and used to support hPS cell culture for 1-2 days (WO 01/51616; Xu et al., Nat. Biotechnol. 19:971, 2001).

Alternatively, fresh non-conditioned medium can be used, which has been supplemented with added factors (like a fibroblast growth factor or forskolin) that promote proliferation of the cells in an undifferentiated form. Exemplary is a base medium like X-VIVO™ 10 (Biowhittaker) or QBSF™-60 (Quality Biological Inc.), supplemented with bFGF at 40-80 ng/mL, and optionally containing stem cell factor (15 ng/mL), or Flt3 ligand (75 ng/mL). These medium formulations have the advantage of supporting cell growth at 2-3 times the rate in other culture systems (WO 03/020920).

Under the microscope, ES cells appear with high nuclear/cytoplasmic ratios, prominent nucleoli, and compact colony formation with poorly discernable cell junctions. Primate ES cells typically express the stage-specific embryonic antigens (SSEA) 3 and 4, and markers detectable using antibodies designated Tra-1-60 and Tra-1-81. Undifferentiated hES cells also typically express the transcription factor Oct-3/4, Cripto, gastrin-releasing peptide (GRP) receptor, podocalyxin-like protein (PODXL), and human telomerase reverse transcriptase (hTERT), as detected by RT-PCR (US 2003/0224411 A1).

By no means does the practice of this invention require that a human blastocyst be disaggregated in order to produce the hES for the practice of this invention. hES cells can be obtained from established lines obtainable from public depositories (for example, the WiCell Research Institute, Madison, Wis. U.S.A., or the American Type Culture Collection, Manassas, Va., U.S.A.). U.S. Patent Publication 2003/0113910 A1 reports pluripotent stem cells derived without the use of embryos or fetal tissue. It may also be possible to reprogram other progenitor cells into hPS cells by using a factor that induces the pluripotent phenotype (Chambers et al., Cell 113:643, 2003; Mitsui et al., Cell 113:631, 2003). Under appropriate conditions, any pluripotent stem cells with sufficient proliferative and differentiation capacities can be used for making dendritic cells according to this invention.

Preparing Dendritic Cells

The antigen presenting cells used in this invention are made by culturing stem cells in an environment that guides the progenitors towards (or promotes outgrowth of) the desired cell type.

In some instances, differentiation is initiated in a non-specific manner by forming embryoid bodies or culturing with one or more non-specific differentiation factors. Embryoid bodies (EBs) can be made in suspension culture: undifferentiated hPS cells are harvested by brief collagenase digestion, dissociated into clusters or strips of cells, and passaged to non-adherent cell culture plates. The aggregates are fed every few days, and then harvested after a suitable period, typically 4-8 days. Specific recipes for making EB cells from hPS cells can be found in U.S. Pat. No. 6,602,711 (Thomson); WO 01/51616 (Geron Corp.); US 2003/0175954 A1 (Shamblott & Gearhart); and US 2003/0153082 A1 (Bhatia, Robarts Institute). Alternatively, fairly uniform populations of more mature cells can be generated on a solid substrate: US 2002/019046 A1 (Geron Corp.).

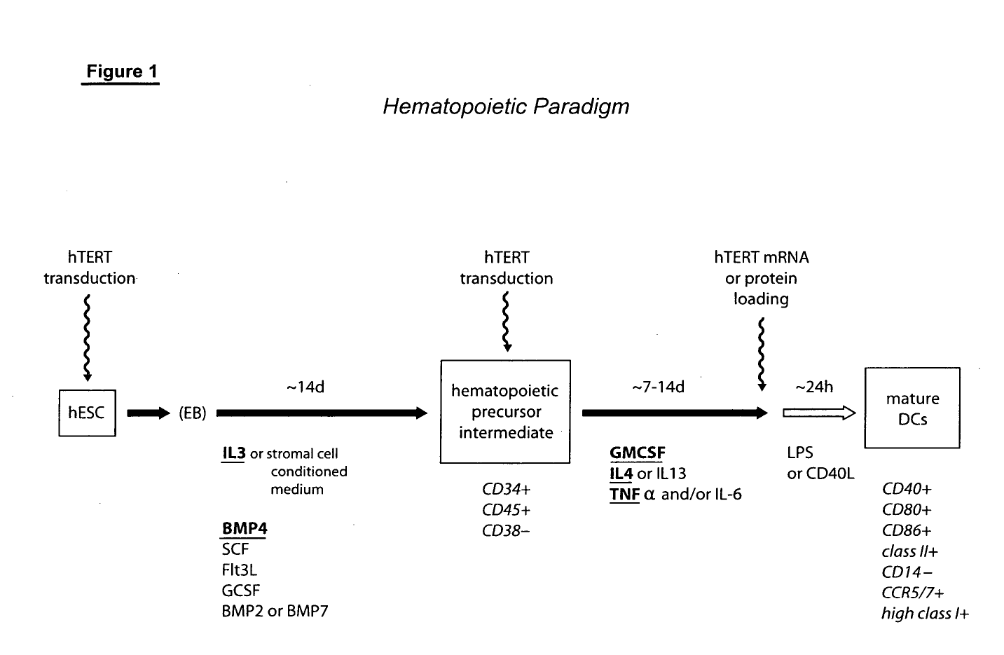

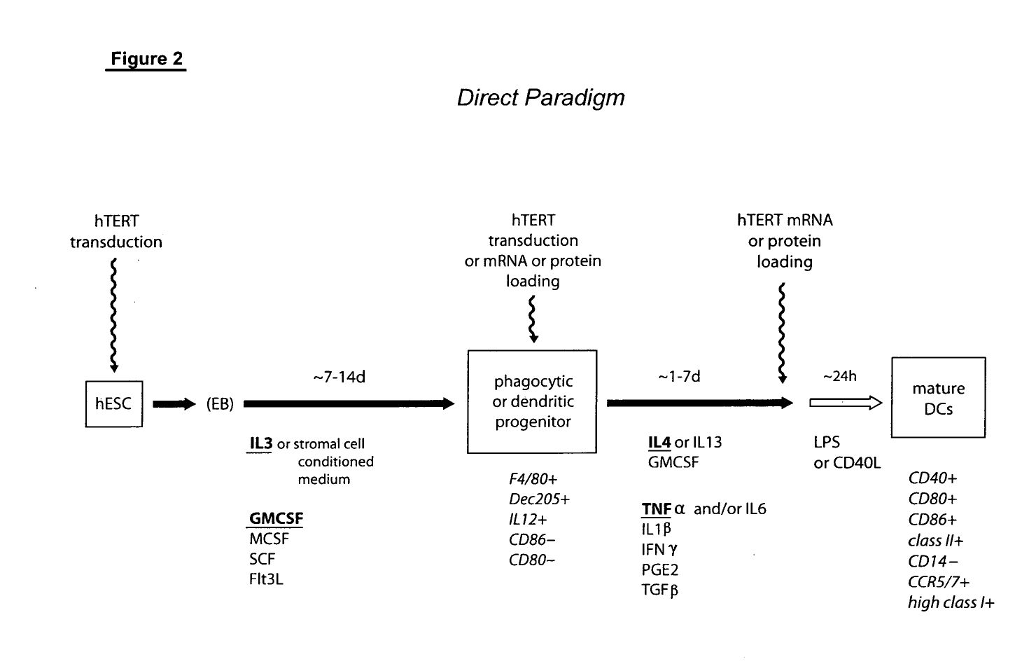

The culture is specifically directed into the dendritic cell lineage by including in the culture medium a factor combination that more specifically promotes the desired phenotype. FIG. 1 and FIG. 2 illustrate two alternative differentiation paradigms.

The Hematopoietic Paradigm (FIG. 1) involves forming an intermediate cell (either as an isolated cell type or in situ) that has features of multipotent hematopoietic precursor cells. Such features may include positive staining for hematopoietic markers CD34 and CD45, and negative staining for CD38. The cells may also have the ability to form colonies in a classic CFU assay.

First-stage hematopoietic differentiation is accomplished by culturing with hematopoetic factors such as interleukin 3 (IL-3) and bone morphogenic protein 4 (BMP4), optionally in combination with other supporting factors such as stem cell factor (SCF), Flt-3 ligand (FIt3L), granulocyte colony stimulating factor (G-CSF), other bone morphogenic factors, or monocyte conditioned medium.

The medium used for differentiating the cells is formulated for or compatible with hematopoietic cell cultures, having components such as isotonic buffer, protein nutrient (serum, serum replacement, albumin, and/or amino acids like glutamine), lipid nutrient (serum lipids, fatty acids, or cholesterol as artificial additives or the HDL or LDL extract of serum), growth promoting hormones like insulin or transferrin, nucleosides or nucleotides, pyruvate, a sugar source (such as glucose), selenium, a glucocorticoid (such as hydrocortisone), or a reducing agent (such as β-mercaptoethanol). Exemplary are X-VIVO™ 15 expansion medium (commercially available from Biowhittaker/Cambrex), and Aim V (Invitrogen/Gibco). See also WO 98/30679 (Life Technologies Inc.) and U.S. Pat. No. 5,405,772 (Amgen).

In addition or as a substitute for some of these factors, hematopoietic differentiation can be promoted by coculturing with a stromal cell lineage (such as mouse lines OP9 or Ac-6, commercially available human mesenchymal stem cells, or the hES derived mesenchymal cell line HEF1 (U.S. Pat. No. 6,642,048). Where the dendritic cells are intended for use in human patients, it may be preferable to avoid contact with other cell types, particularly non-human cell lines. With this in mind, a similar effect can be accomplished by preconditioning the medium by culturing the stromal cells alone, and then using the conditioned medium with the hPS cells in the differentiation protocol.

Subsequently, the hematopoietic intermediate is further differentiated into antigen presenting cells or dendritic cells that may have one or more of the following features in any combination: CD40 positive, CD80 and/or CD83 positive, CD86 positive, Class II MHC positive, highly positive for Class I MHC, CD14 negative, and positive for chemokine receptors CCR5 and CCR7. This can be accomplished by culturing with factors such as granulocyte monocyte colony stimulating factor (GM-CSF), IL-4 or IL-13, a pro-inflammatory cytokine such as TNFα or IL-6, and interferon gamma (IFNγ). Without intending to be limited by theory, it is believed that GM-CSF helps guide the cells towards immunostimulatory (non-toleragenic) cells; IL-4 or IL-13 steer toward dendritic cells and away from macrophages, and TNFα or IL-6 push dendritic cell maturation.

The Direct Paradigm (FIG. 2) is a multi-step process that is designed to direct cells towards the phagocytic or dendritic cell subset early on. Intermediate cells may already bear hallmarks of monocytes ontologically related to dendritic cells or phagocytic antigen presenting cells, and may have markers such as cell surface F4/80 and Dec205, or secreted IL-12. They need not have the capability of making other types of hematopoietic cells. They are made by using IL-3 and/or stromal cell conditioned medium as before, but the GM-CSF is present in the culture concurrently.

Maturation of the phagocytic or dendritic cell precursor is achieved in a subsequent step: potentially withdrawing the IL-3, but maintaining the GM-CSF, and adding IL-4 (or IL-13) and a pro-inflammatory cytokine. Other factors that may be helpful at this stage are IL-1β, interferon gamma (IFNγ), prostaglandins (such as PGE2), and transforming growth factor beta (TGFβ); along with TNFα and/or IL-6 (FIG. 2). A more mature population of dendritic cells should emerge, having some of the characteristics described earlier.

In either the hematopoietic or direct paradigms, it may be beneficial to mature the cells further by culturing with a ligand or antibody that is an agonist for CD40 (U.S. Pat. Nos. 6,171,795 and 6,284,742), or a ligand for a Toll-like receptor (such as lipopolysaccharide or LPS, which is a TLR4 ligand; poly I:C, a synthetic analog of double stranded RNA, which is a ligand for TLR3; Loxoribine, which a ligand for TLR7; or CpG oligonucleotides, synthetic oligonucleotides that contain unmethylated CpG dinucleotides in motif contexts, which are ligands for TLR9)—either as a separate step (shown by the open arrows), or concurrently with other maturation factors (e.g., TNFα and/or IL-6).

In some embodiments of the invention, the cells are divided into two populations: one of which is used to form mature dendritic cells that are immunostimulatory, and the other of which is used to form toleragenic dendritic cells. The toleragenic cells may be relatively immature cells that are CD80, CD86, and/or ICAM-1 negative. Alternatively or in addition, they may be adapted to enhance their toleragenic properties. For example, they can be transfected to express Fas ligand, or inactivated, for example, by irradiation or treatment with mitomycin c.

It will be recognized that the scheme described here is a framework that allows the user to determine various effective factor combinations to make dendritic cells from hPS cells. Each of the process steps will be effective with complex factor mixtures such as those outlined here in detail—but the skilled reader will recognize that not all factors will be critical to generating the desired cell populations. Without undue experimentation, the user may eliminate unnecessary factors and find substitutes by following the phenotypic and functional characteristics of the cells as indicated.

Characteristics of Dendritic Cells

Cells can be characterized according to phenotypic criteria, such as morphological features, detection or quantitation of cell surface or internal markers, or functional activity as stimulators or inhibitors in mixed lymphocyte reactions conducted in culture.

Tissue-specific markers referred to above can be detected using any suitable immunological technique—such as flow immunocytochemistry for cell-surface markers, immunohistochemistry (for example, of fixed cells or tissue sections) for intracellular or cell-surface markers, Western blot analysis of cellular extracts, and enzyme-linked immunoassay, for cellular extracts or products secreted into the medium. A cell population can be assessed as positive for a marker indicated above if at least 25%, 50%, 75%, or 90% of the cells show staining above background, depending on the level of homogeneity required for a particular use. A cell population can be assessed as negative for a particular marker if less than 10% or 5% of the cells show staining, or if the overall level of staining in the population is substantially lower intensity than a positive control, as required.

The expression of tissue-specific gene products can also be detected at the mRNA level by Northern blot analysis, dot-blot hybridization analysis, or by reverse transcriptase mediated polymerase chain reaction (RT-PCR) using sequence-specific primers in standard amplification methods. See U.S. Pat. No. 5,843,780 for further details. Expression of tissue-specific markers as detected at the protein or mRNA level is considered positive if the level is at least 2-fold, and preferably more than 10- or 50-fold above that of a control cell, such as an undifferentiated hPS cell, a fibroblast, or other unrelated cell type.

Antigen presenting cells of this invention are often referred to in this disclosure as “dendritic cells”. However, this is not meant to imply any morphological, phenotypic, or functional feature beyond what is explicitly required. The term is used to refer to cells that are phagocytic or can present antigen to T lymphocytes, falling within the general class of monocytes, macrophages, dendritic cells and the like, such as may be found circulating in the blood or lymph, or fixed in tissue sites. Phagocytic properties of a cell can be determined according to their ability to take up labeled antigen or small particulates. The ability of a cell to present antigen can be determined in a mixed lymphocyte reaction as described. Certain types of dendritic cells and antigen-presenting cells in the body are first identified in tissue sites such as the skin or the liver; but regardless of their origin, location, and developmental pathway, they are considered in the art to fall within the general category of hematopoietic cells. By analogy, the term dendritic cells used in this disclosure also fall in the broad category of hematopoietic cells, whether produced through the hematopoietic or direct paradigm framed earlier, or through a related or combined pathway.

The putative role of hPS derived cells as antigen-presenting cells is provided in this disclosure as an explanation to facilitate the understanding of the reader. However, the theories expostulated here are not intended to limit the invention beyond what is explicitly required. The hPS derived cells of this invention may be used therapeutically regardless of their mode of action, as long as they achieve a desirable clinical benefit in a substantial proportion of patients treated.

Genetic Modifications

In some embodiments of the invention, the cells are permanently transduced with a gene that enables the cells to express the gene product in progeny that bear characteristics of dendritic cells. The cells can be transduced while they are still undifferentiated hPS cells, or at an intermediate stage (such as a hematopoietic or dendritic cell precursor). Methods for genetically altering hPS cells in the presence or absence of feeder cells using lipofectamine are described in US 2002/0168766 A1 (Geron Corp.). Lentiviral and retroviral vectors are also suitable. Alternatively, the expression cassette can be placed into a known location in the genome of the cell by homologous recombination (US 2003/0068818 A1).

Genetic modifications that can promote the immunogenic effect include expression of cytokines such as IL-12 or IL-15 that contribute to cytotoxic T cell activation or memory, or chemokine equivalents such as secondary lymphoid tissue chemokine (SLC), IFNγ (which induces monokine), or lymphotactin (Lptn). Costimulatory molecules like B7 may enhance T cell activation. Inhibition of invariant chain expression (by knockout, antisense, or RNAi technology) may enhance the CD4+ T cell component of the response. The transgene may also cause expression of the target tumor antigen, as described in the next section.

Priming Dendritic Cells to Express Tumor Antigen

The immunogenic dendritic cells are loaded with one or more tumor or tissue specific antigens so as to elicit an immunogenic response against the antigens when administered to a subject. This can be done by pulsing the cells with antigen in peptide or protein form, or genetically altering the cells with a nucleic acid encoding the desired antigen. A cell is said to be “genetically altered” or “transduced” when a nucleic acid (an mRNA, DNA, or polynucleotide vector) has been introduced into the cell, or where the cell is a progeny of the originally altered cell that has inherited the nucleic acid.

An effective method of loading the cells is to combine them with mRNA encoding the antigen of interest. Since mRNA is not stable for extended periods, this is done towards the end of the differentiation protocol or just before administration to the patient. The mRNA can be introduced into the cell by electroporation or cationic lipids (as illustrated in the Example), using cationic peptides (PCT Application by Argos et al., Docket MER028WO), or using dendrimers (Choi et al., Cell Cycle 4:669, 2005; Manunta et al., Nucl. Acids Res. 32:2730, 2004).

The effectiveness of mRNA pulsing is attributed in part to a delay between the time the mRNA is introduced into the cell, and the time by which the protein has been expressed and loaded onto the Class II histocompatibility antigens for presentation. Once the cells are administered, they have time to migrate closer to the tumor site, or to a lymph node through which lymphocytes servicing the tumor are trafficking. Thus, the vaccine can be formulated with the cells preloaded, or the dendritic cells and mRNA can be provided separately, to be combined just before use.

In order to improve the proportion of antigen presented by the dendritic cells to the immune system, it is helpful to design a fusion peptide in which the antigen is conjoined to a protein or peptide sequence that enhances transport into endosomal and other intracellular compartments involved in Class II loading. For example, a suitable heterologous leader or signal sequence for the endosomal compartment can be placed at the N-terminal; and the transmembrane and lumenal component of a member of the LAMP family (U.S. Pat. No. 5,633,234; WO 02/080851; R. Sawada et al., J. Biol. Chem. 268:9014, 1993) can be placed at the C-terminal for lysosomal targeting. Endosomal and lysosomal sorting signals include tyrosine-based signals, dileucine-based signals, acidic clusters, and transmembrane proteins labeled with ubiquitin (Bonifacino et al., Annu. Rev. Biochem. 72:395, 2003; U.S. Pat. No. 6,248,565).

Instead of mRNA, the dendritic cells can be loaded with protein or peptide made by chemical synthesis or by recombinant expression. Again, loading of the cells is done just before administration to the patient. The cells can be supplied preloaded or in conjunction with the loading protein to be combined just before use. In some instances, it may be preferable not to use a peptide that is loaded onto the Class II antigen directly, since it may detach before the cell reaches the cancer site. Instead, the protein can be prepared in a branched, aggregate, or complexed form that requires substantial processing time.

Alternatively, the cells can be treated with nucleic acid vectors encoding the tumor antigen (M. Frolkis et al., Cancer Gene Ther. 10:239, 2003). Exemplary are plasmid/cationic lipid complexes, adenovirus vectors, or cDNA encoding tumor antigen loaded onto dendrimers (generally described by J. W. M. Bulte et al., J. Cerebral Blood Flow Metab. 22:899, 2002; L. M. Santhakumaran et al., Nucl. Acids Res. 32:2102, 2004), or other small particulates that enhance uptake by phagocytic cells

Pulsing of the dendritic cells with mRNA, adenovirus vectors, or protein can be done when the DCs are fully mature. Alternatively (if the cells are phagocytic or otherwise susceptible to antigen loading at an immature stage), the cells can be loaded before or during a final maturation in a maturation cocktail containing proinflammatory cytokines TNFα and/or IL-6, optionally with other factors (FIGS. 1 & 2; Example 1); or with LPS or a CD40 ligand. The loaded cells can then be administered to the subject being treated, or preserved (e.g., by freezing) for later use.

Inducible Antigen Presentation

As an alternative to loading the cells just before use, the cells can be transduced at an earlier stage with an inheritable expression cassette: for example, using a retroviral vector or DNA plasmid. The vector encodes the antigen of interest, optionally conjoined to the transport protein like LAMP, under control of a suitable promoter that drives expression when the cell bears the dendritic cell phenotype. This strategy saves the final loading step, and confers the entire cell line with the capacity to produce the antigen of interest when needed.

As a means for mimicking the pulsing effect with the mRNA, the promoter used in the expression cassette can be a promoter that is inducible at a time analogous to mRNA loading.

Suitable candidates are promoter systems that are inducible with tetracycline (Shockett et al., Proc. Natl. Acad. Sci. USA 92:6522, 1995; Rossi et al., Molec. Cell 6:723, 2000); isopropyl-β-D-thiogalactopyranoside (ITPG) (Liu et al., Biotechniques 24:624, 1998; Li et al., Biotechniques 28:577, 2000); picolinic acid or desferrioxamine (Pastorino et al., Gene Ther. 11:560, 2004); metallothionein (activated by heavy metal ions Zn⁺⁺, Cd⁺⁺, Cu⁺⁺ and Hg⁺⁺: C. H. Yan et al., Biochim. Biophys. Acta 1679:47, 2004); ecdysone (G. H. Luers et al., Eur. J. Cell Biol. 79:653, 2000); and biphenyl compounds (Takeda et al., Biosci. Biotechnol. Biochem. 68:1249, 2004); as well as promoter systems inducible by heat shock, light, or radiation (D. W. Cowlinget al., PNAS 82:2679, 1985; S. Shimizu-Sato et al., Nat. Biotechnol. 20:1041, 2002; J. Worthington et al., J. Gene Med. 6:673, 2004). Illustrations have been published for transfecting cells with transgenes under control of inducible promoters using lentiviral vectors (Kafri et al., Mol. Ther. 1:516, 2000; Vigna et al., Mol. Ther. 5:252, 2002) or AAV vectors (Apparailly et al., Hum. Gene Ther. 13:1179, 2002; Charto et al., Gene Ther. 10:84, 2003).

To employ this embodiment of the invention, cells are genetically modified as undifferentiated hPS cells, or at a progenitor stage when the cells can still replicate. After introduction of the transgene having the antigen encoding region under control of the inducible promoter, the cells are expanded and further differentiated in the absence of the inducing compound, leaving the expression cassette inactive.

Just before administering to the subject, the cells are combined with the corresponding inducing compound (e.g., tetracycline) to initiate expression of the target tumor antigen. The time required for transcription and Class II loading of the gene product will then give the cells time to traffic to an effective location near the cancer site. The cells may also be matured, activated or excited in some fashion (e.g., with LPS or a ligand for CD40) so as to mimic the promotional effect that apparently ensues from the electroporation procedure when the cells are loaded with mRNA.

Choice of Tumor Antigen

An exemplary antigen for presentation by the dendritic cells of this invention is telomerase reverse transcriptase (U.S. Pat. No. 6,261,836; GenBank Accession No. AF015950). TERT is particularly suitable because ˜90% of tumors of all types upregulate telomerase activity. Telomerase expression overcomes replicative senescence that otherwise prevents most adult cell types from exceeding more than about 50 cell divisions. Telomerase restores telomere repeats at the ends of chromosomes, allowing the cells to replicate indefinitely. By using TERT (the catalytic component of telomerase) as the immunogen, the vaccine targets the key enzyme that the tumor requires to remain immortal.

In this embodiment, the dendritic cell vaccine is primed to present one or more immunogenic epitopes of TERT, typically of human origin (SEQ. ID NO:1). Immunogenic epitopes can be identified by analyzing the TERT sequence using a suitable algorithm, available from the BioInformatics & Molecular Analysis website (K. C. Parker et al., J. Immunol. 152:163, 1994), or from ProImmune Advanced Solutions (MHC Ligands and Peptide Motifs, by H. G. Rammensee et al., Chapman & Hall, 1998) in combination with empirical tests in preclinical or clinical trials.

Of course, the use of one immunogenic epitope of TERT in the composition is all that is needed, but the vaccine will typically contain 100, 200, 500, or 1000 consecutive amino acids of the human TERT sequence comprising multiple epitopes, up to the full length of the protein (1132 amino acids). Since the role of TERT in this system is to act as an immunogen, and not to immortalize the antigen-presenting cells, it may be desirable to use a non-functional form of TERT that either does not bind telomerase RNA component, or which lacks telomerase catalytic activity when associated with telomerase RNA component.

Inactive forms can be generated by supplying TERT in fragmented form (say, a fragment or cocktail of fragments of 100 or 200 consecutive amino acids at a time). Inactive forms can also be made by deleting or mutating part of the TERT sequence required for RNA binding or catalytic activity, such as in the conserved motif regions: see U.S. Pat. Nos. 6,610,839 and 6,337,200; and WO 98/14592. In this way, the protein will be devoid of telomerase catalytic activity in the presence of telomerase RNA component.

Other modifications to the TERT sequence can be made for epitope enhancement: either to increase affinity for MHC molecules, or to increase T cell receptor triggering, or to inhibit proteolysis of the peptide by serum proteases. Methods of epitope enhancement are described generally in J. A. Berzofsky, Ann. N.Y. Acad. Sci. 690:256, 1993; S. A. Rosenberg et al., Nat. Med. 4:321, 1998; L. Rivoltini et al., Cancer Res. 59:301, 1999; L. H. Brinckerhoff et al., Int. J. Cancer 83:326, 1999; and L. Fong et al., Proc. Natl. Acad. Sci. USA 98:8809, 2001. It is understood that protein sequences incorporating such variations are equivalent to the prototype sequences from which they were derived, for the general use of tumor target antigens in the context of this invention.

Further information on the structure and function of telomerase and its role in cancer can be found in Telomerases, Telomeres and Cancer (Molecular Biology Intelligence Unit, 22) by G. Krupp & R. Parwaresch, Kluwer Academic Publ., 2002; and Telomeres and Telomerase: Methods and Protocols by J. A. Double & M. J. Thompson, Humana Press, 2002. Other publications on telomerase include U.S. Pat. No. 6,440,735 (Geron Corp.); EP 1093381 B1 (GemVax); WO 00/61766; WO 01/60391; WO 03/038047; and WO 04/002408; G. Morin, J. Natl. Cancer Inst. 87:857, 1995; C. Harley et al., Gen. Dev. 5:249, 1995; S. P. Lichtsteiner et al., Ann. N.Y. Acad. Sci. 886:1, 1999; R. H. Vonderheide et al., Immunity 10:673, 1999; R. H. Vonderheide et al., Clin. Cancer Res. 10:828, 2004; M. Greener, Mol. Med. Today 6:257, 2000; S. Saeboe-Larssen et al, J. Immunol. Meth. 259:191, 2002; Z. Su et al., Cancer Res. 62:5041, 2002; M. Frolkis et al., Cancer Gene Ther. 10:239, 2003; E. Sievers et al., J. Urol. 171:114, 2004; J. Hernández et al., Proc. Natl. Acad. Sci. USA 99:12275, 2002; B. Minev et al., Proc. Natl. Acad. Sci. USA 97:4796, 2000; S. Nair et al., Nature Med. 6:1011, 2000; and A. Heiser et al., Cancer Res. 61:3388, 2001.

Telomerase reverse transcriptase from other species (US 2004/0106128 A1), and other telomerase-associated proteins may also be used as tumor antigen. See, for example, U.S. Pat. No. 6,300,110 (TPC2 and TPC3), U.S. Pat. No. 6,277,613 (Tankyrase I), U.S. Pat. No. 6,559,728 (Tankyrase II), U.S. Pat. No. 5,981,707 (TP1), WO 98/23759 and WO 99/51255.

Alternatively or in addition, the dendritic cells of this invention can be primed to present selected targets other than telomerase that are critical to the survival and proliferation of the cancer cell in vivo. Other antigens of interest include:
Tissue-specific antigens that are elevated in cancer, such as carcinoembryonic antigen (CEA, colorectal cancer); α-fetoprotein (liver cancer); prostate cancer antigen (PSA, prostate cancer), mitochondrial creatine kinase (MCK, muscle cancers), myelin basic protein (MBP, oligodendrocyte specific), glial fibrillary acidic protein (GFAP, glial cell specific), tyrosinase (melanoma), and neuron cancer enolase (NSE, neuronal cancers).
Mutated forms of tumor suppressor genes, such as K-ras (colorectal carcinomas), and p53 (˜65% of all cancers). J. L. Bos, Cancer Res. 49:4682, 1989; Chiba et al., Oncogene 5:1603, 1990.
Viral proteins expressed by virally induced cancers, such as human papillomavirus 16/18 E6 and E7 proteins (cervical cancer) or Epstein Barr Virus peptides (EBV, B cell malignancies).
Tumor-specific antigens such as MART-1 (melanoma), gp100 (melanoma), HER2/neu (breast and epithelial cancers); NY-ESO-1 (testes and various tumors), Thymus-leukemia antigen (TL), and proteins of the MAGE family (hepatocellular cancer and other tumors).
Survivin and other apoptosis inhibiting proteins expressed preferentially by tumor cells (M. Zeiss et al., J. Immunol. 170:5391, 2003).
Components involved in angiogenesis, such as vascular endothelia growth factor (VEGF, expressed in angiogenic stroma and tumor cells), VEGF receptor 2, Id1, Id3, and Tie-2 (preferentially expressed during neoangiogenesis) (US 2004/0115174 A1).

General reviews for tumor related antigens useful as cancer vaccine targets include the text Tumor Antigens Recognized by T Cells and Antibodies by H. J. Stauss, Y. Kawakami, & G. Parmiani, CRC Press, 2003; and articles by Rosenberg, Immunity 10:281, 1999; Nestle et al., Nat. Med. 4:328, 1998; Dermime et al., Br. Med. Bull. 62:149, 2002; and Berzofsky et al., J. Clin. Invest. 113:1515, 2004. Methods for identifying additional cancer target antigens are described in Barnea et al., Eur J Immunol. 32:213, 2002; Schirle et al., Eur J Immunol. 30:2216, 2000; Vinals et al., Vaccine 19:2607, 2001; Perez-Diez et al., Cell. Mol. Life Sci. 59:230, 2002; Radvanyi et al., Int. Arch. Allergy Immunol. 133:179, 2004.

The use of this invention is not limited to tumor targets disclosed here or already in the published literature. New tumor antigens can be identified empirically, for example, by using mRNA produced by tumor cells to stimulate lymphocytes and make target cells for an in vitro cytotoxicity assay, and amplifying up cDNA from lysed target cells. See U.S. Pat. No. 6,387,701 (Nair et al.).

Use of hPS Derived Dendritic Cells to Induce an Immune Response

The dendritic cells of this invention can be used to induce an immune response against the antigen of interest, and/or to treat cancer in a patient.

Modes of Therapy

One method of using this invention in therapy is to prepare mature dendritic cells and load them with antigen: either by pulsing with tumor antigen in the form of protein or nucleic acid, or by inducing the promoter of an antigen expressing transgene by combining with an inducing compound. After maturation and loading, about 1×10⁷ cells can be administered intradermally in ˜200 μL isotonic saline, and repeated as necessary to prime or maintain the response.

In another approach, the composition contains not mature dendritic cells, but cells expressing an earlier phenotype (e.g., Dec 205 positive, F4/80 positive, or IL-12 positive, but CD80 or CD86 negative). The cells are loaded with antigen as already described, administered in the precursor form, and allowed to mature in vivo. To enhance or accelerate maturation of cells in the patient, the site of administration can be treated previously or simultaneously with an immunomodulating maturation-promoting adjuvant, such as imiquimod cream (Aldara®; commercially available from 3M Corp.); or polyarginine.

When a dendritic cell composition of this invention is used as an off-the-shelf pharmaceutical, there may be a histocompatibility mismatch between the cells in the preparation and the patient being treated. In some instances, mismatch at the Class II loci may enhance the effect of the vaccine. Allogeneic cells can cross-feed host antigen presenting cells by way transferring packaged tumor antigen to them in the form of exosomes (S. L. Altieri et al., J. Immunother. 27:282, 2004; F. Andre et al., J. Immunol. 172:2126, 2004; N. Chaput et al., Cancer Immunol. Immunother. 53:234, 2004). If the administered cells are taken up instead by phagocytic cells in the host, their tumor antigen payload will be presented by the host cells as a matter of course.

In other instances, HLA mismatch may dampen the effect of the vaccine—either by promoting premature elimination of the cells (especially after multiple administration), or by generating a strong anti-allotype response that distracts the immune system from the intended target. In this context, it may be advantageous to use a vaccine preparation in which at least some of the HLA Class I alleles on the dendritic cells (especially at the A2 locus) are shared with the patient. In this way, at least some of the tumor target antigen will be presented in autologous Class I molecules, enhancing the anti-tumor response and diminishing the allo response.

Partial match can be achieved simply by providing a dendritic cell vaccine made of a mixture of cells bearing two or more of the common HLA-A allotypes (HLA-A2, A1, A19, A3, A9, and A24). Complete match for most patients can be achieved by providing the clinician with a battery of different dendritic cells from which to select, each possibly bearing only a single allotype at the HLA-A locus. HLA homozygous dendritic cells can be made from hPS cells genetically modified to knock out the second allele, or from hPS cells derived from a blastocyst that was homozygous at the HLA-A locus. Treatment would involve identifying one or more HLA allotype(s) in the patient by standard tissue typing, and then treating the patient with dendritic cells having HLA allotype(s) that match those of the patient. For example, a patient that was HLA-A2 and A19 could be treated with either HLA-A2 or HLA-A19 homozygous cells, or with a mixture of both.

Potential negative effects of HLA mismatch can also be dealt with by generating immune tolerance against the foreign allotypes. During preparation of the vaccine, the hPS cells are divided into two populations: one for generating immature toleragenic dendritic cells, and the other for generating mature dendritic cells for antigen presentation. Because they are derived from the same line, the toleragenic cells are designed to induce HLA-specific tolerance that will enhance graft acceptance of the mature cells. The subject first receives one or more administrations of the toleragenic cells to generate a sufficient degree of immune unresponsiveness (measurable, for example, in a mixed lymphocyte reaction). Once tolerance is in place (a week to a month later), the subject is then administered with the antigen-loaded dendritic cells as often as needed to elicit the immune response against the target tumor antigen.

In whatever manner the vaccine is administered, it will generally take multiple administrations to achieve a substantial immune response against a self-antigen. The practice of this invention may employ a course of two, three, six, or more administrations of the dendritic cell vaccine on a periodic schedule (e.g., weekly or biweekly). Once a sufficient level of immunity has been achieved to achieve clinical benefit, maintenance boosters may be required, but can generally be given on a less frequent basis (e.g., monthly or semi-annually).

Combination Therapies

Treatment with the dendritic cell vaccines of this invention can be conducted concurrently or sequentially with other vaccine types directed to the same tumor target. For example, the patient can be administered with a course of up to about 6 weekly vaccinations with a dendritic cell expressing human TERT, in order to establish a cytotoxic lymphocyte response sufficient to control tumor progression, as illustrated in the example below. After priming a memory response in this way, the response is then maintained by occasional administration with a non-cellular vaccine against TERT, such as an adenovirus or plasmid vector expressing immunogenic epitopes of SEQ. ID NO:1.

The vaccines of this invention can also be used in conjunction with other technologies that improve the immunization effect or otherwise serve as an adjunct to therapy for the cancer. For example, the subject can be treated simultaneously and/or in advance with an antibody, aptamer, or other compound that inhibits CTLA-4 (S. Aantulli-Marotto et al., Cancer Res. 63:7483, 2003). This can help minimize innate tolerance to the tumor target, potentiating the response to the vaccine. Inhibition of invariant chain expression in dendritic cells can help stimulate CD4+ T-cell responses and tumor immunity (Y. Zhao et al., Blood 102:4137, 2003; WO 04/016803). It is also possible to increase the presentation of a peptide on a mammalian cell, by inhibiting activity of an MHC class I pathway-associated component, such as a TAP protein or a proteasome, before loading antigen. This can be done by introducing into the cell an antisense oligonucleotide that is complementary to mRNA encoding a TAP protein, or by contacting the cell with a competitive inhibitor of a proteasome (U.S. Pat. No. 5,831,068).

It is a premise of this invention that dendritic vaccines can also be used to potentiate the effect of other treatments for cancer. This includes standard treatment such as chemotherapy or radiation, and other therapies that are specific to a particular type of cancer. One such embodiment of the invention is hTERT based dendritic cell vaccines, made from hPS cells as already described, or from normal PBMCs (e.g., Gilboa and Vieweg, Immmunol. Rev. 199:251, 2004); in combination with another telomerase specific therapy: particularly oncolytic or other tumor killing viral vectors driven by the hTERT promoter (U.S. Pat. Nos. 6,610,839 and 6,713,055; EP 1147181 B1); or oligonucleotides that inhibit telomerase by complexing with the RNA component (U.S. Pat. No. 6,608,036; S. Gryaznov et al., Nucleosides Nucleotides Nucl. Acids 22:577, 2003). The patient is treated with the two therapeutic agents simultaneously or sequentially: for example, a short course of vector or oligonucleotide therapy to eradicate tumor cells; in combination with hTERT immunization beginning at about the same time, and repeated on a regular schedule to prevent recurrence.

Preclinical Testing

Before implementation for human therapy, the user of this technology may wish to test the vaccine components both in vitro and in an appropriate animal model.

Tissue culture assays for antigen presentation can be conducted in several different ways. For example, T lymphocytes are isolated from the peripheral blood of a normal human donor bearing the HLA-A2 allotype. hES derived dendritic cells are made from an HLA-A2 positive hES cell line, pulsed with hTERT mRNA, inactivated, and cultured with the matched T cells in the presence of IL-2. After ˜5 days, the T cells are harvested, and a standard ⁵¹Cr release assay is performed using HLA-A2 positive hTERT loaded T2 cell targets, or HLA-A2 allotype tumor cells. Specific lysis or cytokine secretion measured by ELISPOT (IL-2 or IFNγ from Th1 cells; IL-4 or IL-5 from Th2 cells) correlates with effectiveness of the hES derived dendritic cells to present antigen and stimulate the responder T cells.

In another example, tumor cells and post-immunization PBMCs are recovered from a patient undergoing therapy with autologous hTERT pulsed dendritic cells. hES derived dendritic cells of this invention are pulsed with hTERT mRNA, and used to stimulate T lymphocytes isolated from the patent PBMCs. The T cells are then assayed for cytotoxicity against matched tumor cell targets from the same patient. ⁵¹Cr release or cytokine secretion again correlates with effectiveness of the hES derived dendritic cells to present antigen.

Animal models can be conducted using hES derived dendritic cells pulsed with hTERT to treat human tumors; or mouse TERT to treat C57BL/6 mouse tumors (WO 2004/002408). Immune status can be evaluated by obtaining peripheral blood mononuclear cells from the treated animals, and isolating CD4+ and CD8+ T cells for an IFNγ ELISPOT assay (D. I. Stott, J. Immunoassay 21:273, 2000). 1×10⁵ T cells are cultured overnight with 1×10⁴ antigen-expressing dendritic cells in wells of a microtiter plate precoated with IFNγ capture antibody. Labeled IFNγ detection antibody is then added, and the IFNγ released is measured as an indicator of active antigen-specific T cells in the peripheral blood. Functional specificity can be confirmed by cytolysis assay using ⁵¹Cr labeled antigen loaded dendritic cell targets.

Therapeutic Use

The pharmaceutical compounds of this invention can be used in therapy to achieve any desirable clinical result. Patients having tumors known or suspected to express the tumor antigen (about 90% of tumors in the case of hTERT) are treated with a dendritic cell vaccine according to this invention loaded with the corresponding tumor antigen. This application also contemplates the use of tumor antigen expressing cells for prophylactic purposes for high-risk patients having a genetic predisposition for certain tumor types, or a prior history of cancer. Other life compromising conditions that would benefit by immunization (for example, against a viral or bacterial pathogen) can be treated using the dendritic cell vaccines of this invention, in which the target antigen is loaded into or expressed by the cells. The making of such vaccines follows from this description mutatis mutandis, using target antigen (e.g., viral or bacterial epitopes) in place of tumor antigen.

The immunological effect can be evaluated using assays for measuring specific T lymphocyte response, such as ELISPOT, mixed lymphocyte reactions, and cytolytic assays as already described. Therapeutic effect can be evaluated by standard clinical criteria appropriate for the condition. For some tumor types, serum level of a tumor related antigen (like PSA) can be used as a proxy for growth or activity of the tumor. Desirable outcomes include regression of the tumor mass, or at least a slowing in the rate of growth or in the formation of metastasis, improved survival rate, and improved quality of life. Ultimate choice of the treatment protocol, dose, and monitoring is the responsibility of the managing clinician.

Published information relating to the manufacture of dendritic cells and their use in therapy can be found in U.S. Pat. No. 5,962,320 (Robinson, Stanford); U.S. Pat. No. 6,121,044 (Dendreon); U.S. Pat. No. 6,306,388 (Nair et al.); U.S. Pat. No. 6,387,701 (Nair et al.); U.S. Pat. No. 6,440,735 (Geron Corp.); and U.S. Pat. No. 6,475,483 (Steinman et al., Merix); US 2004/0072347 A1 (B. Schuler-Thurner et al.); D. Boczkowski et al., J. Exp. Med. 184:465, 1996; E. Maraskovsky et al., Blood 96:878, 2000; C. Klein et al., J. Exp. Med. 191:1699, 2000; A. Heiser et al., Cancer Res 61:3388, 2001; S. Saeboe-Larssen et al, J. Immunol. Meth. 259:191, 2002; S. K. Nair et al., Eur. J. Immunol. 27:589, 1997; S. K. Nair et al., Nat. Med. 6:1011. 2000; L. Ping et al., J. Leuko. Biol. 74:270, 2003; E. Sievers et al., J. Urol. 171:114, 2004; R. H. Vonderheide et al., Clin. Cancer Res. 10:828, 2004; Z. Su et al., Cancer Res. 62:5041, 2002; Z. Su et al., Cancer Res. 63:2127, 2003; Ardavin et al., Immunity 20:17, 2004; H. W. Chen et al., Int. Immunol. 15:427, 2003; F. Sallusto et al., J. Exp. Med. 179:1109, 1994; J. Banchereau et al., Cancer Res. 61:6451, 2001; A. Mackensen et al., Int. J. Cancer 86:385, 2000; M. Rosenzwajg et al., J. Leukoc. Biol. 72:1180, 2002; M. S. Labeur et al., J. Immunol. 162:168, 1999; M. V. Dhodapkar et al., Blood 100:174, 2002; L. Fong et al., Proc. Natl. Acad. Sci. USA 98:8809, 2001; E. Gilboa & J. Vieweg, Immmunol. Rev. 199:251, 2004; and Su et al., J. Immunol. 174:3798, 2005.

### Commercial Embodiments

When intended for clinical use, the dendritic cell preparations described in this disclosure are formulated for administration to a human subject. This means that the cells are prepared in compliance with local regulatory requirements, are sufficiently free of contaminants and pathogens for human administration, and are suspended in isotonic saline or other suitable pharmaceutical excipient.

For general principles in medicinal formulation and use of cellular vaccine compositions, the reader is referred to Handbook of Cancer Vaccines by M. A. Morse et al., Humana Press, 2004; Cancer Vaccines and Immunotherapy by P. L. Stern et al., eds., Cambridge Univ. Press, 2000; and the most recent edition of Good Manufacturing Practices for Pharmaceuticals by S. H. Willig, Marcel Dekker. The testing and use of dendritic cell vaccines is reviewed in the reference texts Dendritic Cell Protocols (Methods in Molecular Medicine, 64) by S. P. Robinson et al., Humana Press, 2001; and Dendritic Cells in Clinics by M. Onji, Springer-Verlag, 2004.

Any of the dendritic cell preparations of this invention (precursors or mature, immunogenic or toleragenic, and if immunogenic, before or after loading with antigen) can be stored after preparation to be used later for therapeutic administration or further processing. Methods of cryoconserving dendritic cells both before and after loading are described in PCT publication WO 02/16560 (B. Schuler-Thurner et al.).

Occasional reference to a pharmaceutical composition in this disclosure as a “vaccine” implies no particular mode of action or administration. The term means only that it has been formulated for administration to a human subject as already described. A vaccine may be designed as an immunogenic composition for generating a CTL response against a target tumor antigen—but this need not be demonstrated as long as the composition is therapeutically effective according to any suitable clinical criterion in a reasonable proportion of treated cancer patients.

Various cell preparations of this invention can be maintained or supplied in combination with each other or with materials useful in their manufacture or use. Commercial embodiments include any system or combination of cells or reagents that exist at any time during manufacture, distribution, testing, or clinical use of the hPS derived dendritic cells, as described in this disclosure. Cell populations that may be useful together are undifferentiated hPS cells, hPS-derived dendritic cell precursors, mature dendritic cells, toleragenic dendritic cells, or other differentiated cell types, in any combination, sometimes derived from the same hPS cell line.

Other embodiments comprise the dendritic cells in combination with the factor(s) effective to load them with tumor antigen (e.g., TERT peptide, TERT encoding mRNA, or other tumor antigen); promoter inducing compound(s); factor(s) effective to prime the cells; factors for administration to the subject so as to optimize the immunization; or any useful combination of such reagents or factors. Combinations of cells and/or reagents may be packaged together in kit form, or in separate containers in the same facility, or at different locations, at the same or different times, under control of the same entity or different entities sharing a business relationship.

The composition(s) and combinations of this invention may be packaged in a suitable container with explicit written instructions for a desired purpose, such as vaccinating a subject, eliciting an anti-TERT or anti-tumor immunological response, or treating a cancer, as exemplified elsewhere in this disclosure.

### The Following Example is not Intended to Limit the Claimed Invention

### EXAMPLE

Use of hTERT Dendritic Cell Vaccine to Treat Prostate Cancer

This example shows results obtained from an ongoing Phase I/II clinical trial designed to test the safety and efficacy of a dendritic cell vaccine targeting human telomerase reverse transcriptase, made from autologous peripheral blood cells. The patients were treated by Dr. Johannes Vieweg's group at the Duke University Medical Center in North Carolina, in conjunction with Merix Bioscience. Laboratory experiments and data analysis in support of the trial are being conducted both at Duke and at Geron Corporation.

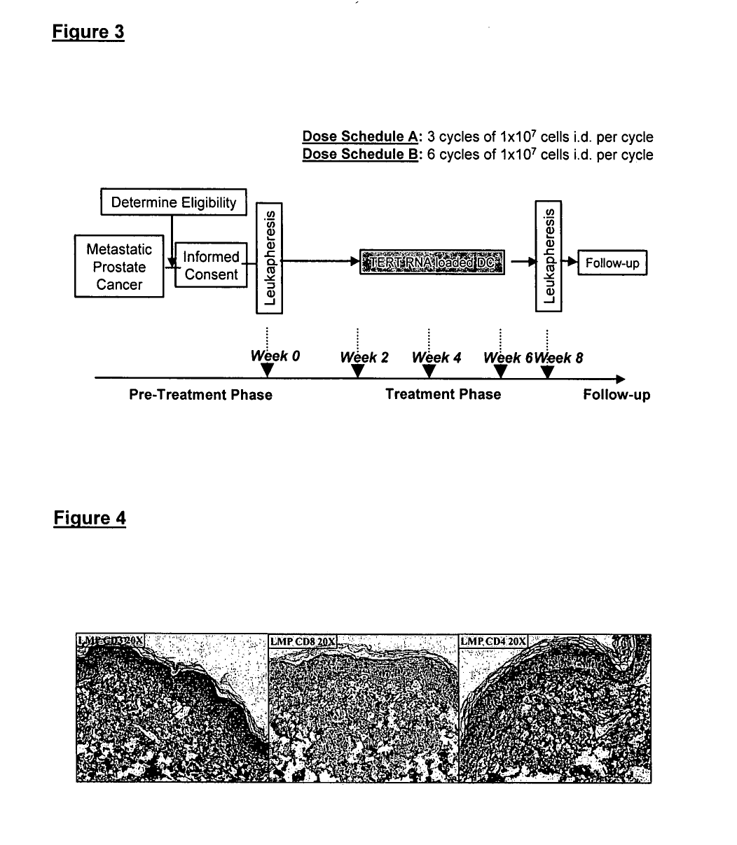

FIG. 3 is an overview of the trial design. Autologous dendritic cells were generated by culturing peripheral blood mononuclear cells from the patient with recombinant human GM-CSF (800 U/mL) and IL-4 (500 U/mL) in X-VIVO™ 15 medium for 7 days. (1000 U/mL of both GM-CSF and IL-4 were used in some subsequent experiments). The cells were then transfected via electroporation with mRNA encoding human TERT.

The RNA was generated by in vitro transcription of a plasmid encoding full-length TERT, under control of the bacteriophage T7 promoter (a standard constitutive promoter often used in vectors of this kind). In some cases, the hTERT cDNA (pGRN145 plasmid; ATCC Accession No. AF01595) was modified by replacing 167 amino-terminal amino acids with amino acids 1-27 of human gp96 (an endosomal leader sequence); and replacing the hTERT stop codon with amino acids 383-416 of human LAMP-1, comprising the transmembrane region and lysosomal targeting sequence. mRNA was generated from linearized plasmids using bacteriophage T7 RNA polymerase, generating hTERT mRNA of 3528 nucleotides, and hTERT/LAMP-1 RNA of 3225 nucleotides (Z. Su et al., Cancer Res. 62:5041, 2002).

Transfection of dendritic cells with hTERT or LAMP hTERT mRNA was performed by electroporation. Briefly, washed cells were suspended in Viaspan® medium (Barr Laboratories, Pomona, N.Y.) at 4×10⁷ cells per mL. They were then co-incubated for 5 min with 1 μg RNA per 10⁶ cells on ice and electroporated in 0.4 cm cuvettes by exponential decay delivery at 300 V and 150 μF. See also V. F. Van Tendeloo et al., Blood 98:49, 2001. As an alternative, the RNA can be delivered into the cells using a cationic lipid such as N-[1-(2,3-dioleoyloxy)propyl]-N,N,N-trimethylammonium methylsulfate (DOTAP; Roche) (Z. Su et al., supra).

After electroporation, the cells were centrifuged, resuspended in X-VIVO™ 15 medium, and matured for 20 h with 10 ng/mL TNFα, 10 ng/mL IL-1β, 150 ng/mL IL-6, and 1 μg/mL IL-6 (H. Jonuleit et al., Eur. J. Immunol. 27:3135, 1997). (In some subsequent experiments, the medium also contained 800 U/mL GM-CSF, 500 U/mL IL-4, and 100 ng/mL PGE2.) Cells had the following phenotype: Lin negative, HLA Class I and Class II high, CD3 negative, CD14 low, CD80 low, CD86 high, and CD83 high, consistent with mature, monocyte-derived DCs. They were cryopreserved in heat-inactivated autologous plasma supplemented with 10% DMSO and 5% glucose until use.

Patients with metastatic prostate cancer, clinical stages D1-D3 were recruited into the study. They were administered intradermally with 1×10⁷ TERT-pulsed dendritic cells in 200 μL saline, either every other week (3 times) or every week (6 times) over the course of six weeks of therapy.

FIG. 4 shows the delayed-type hypersensitivity (DTH) reactions observed at the injection site following intradermal administration of the dendritic cells to the patients. Immunocytochemical analysis of cells present at the DTH reaction sites show the presence of both CD8 and CD4 T lymphocyte subsets beginning at vaccine cycle two. This is consistent with rapid onset of an antigen-specific cellular immune response, comprising both cytotoxic effector cells, and cells that mediate a Type IV hypersensitivity reaction.

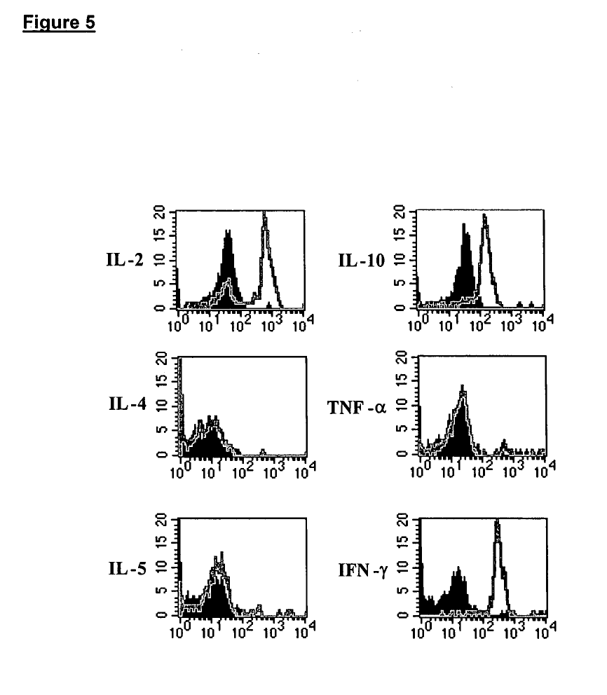

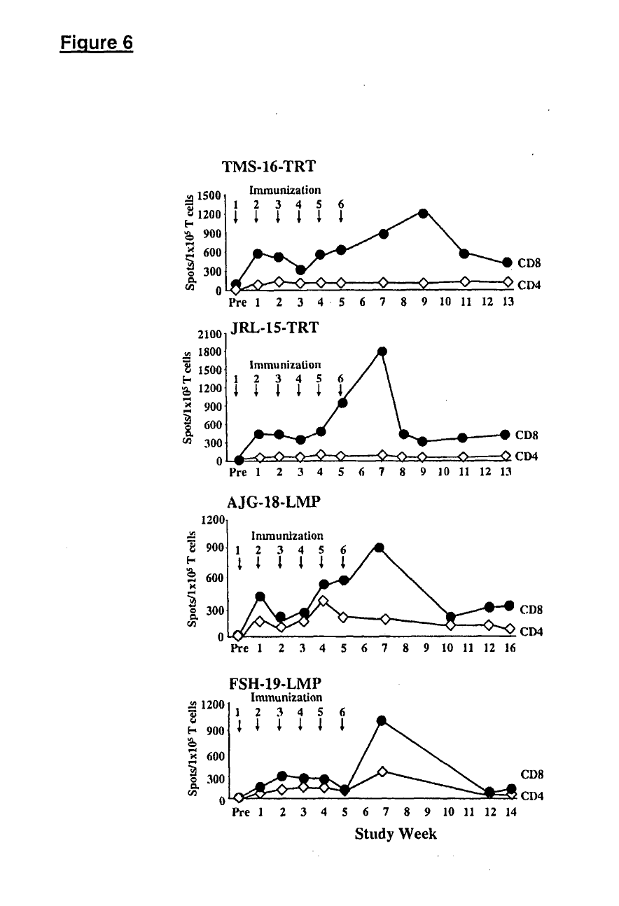

FIG. 5 shows cytokine expression profiles of vaccine-induced TERT-specific CD4+ T lymphocytes. The cells were isolated by magnetic bead separation from peripheral blood after treatment. Expression of the cytokines was analyzed using a cytometric bead array assay. The results show antigen-specific secretion of the cytokines IL-2, IL-10, and IFNγ, which is consistent with stimulation of a Th-1 type antigen-specific cellular immune response.

FIG. 6 shows generation of telomerase-specific cytotoxic T cells during and following vaccination. Some patients were treated with dendritic cells transfected with mRNA encoding human telomerase reverse transcriptase alone (abbreviated here as TRT). Others were treated with the same sequence conjoined to a LAMP trafficking signal peptide (LMP). Peripheral blood cells were collected, stimulated with TERT-RNA transfected antigen presenting cells, and the proportion of cells co-expressing CD8 and IFNγ was measured. Antigen specific lytic activity of these cells was subsequently demonstrated in a standard ⁵¹Chromium release assay.

The results show that CD8+ antigen-specific cytotoxic T cells are present in the circulation as early as one week after the first vaccination. After the sixth injection, the level climbed to about 2,000 per 10⁵ cells (about 2% of the total pool of circulating T cells).

This is quite remarkable and unexpected. Since TERT is encoded in the human genome and expressed in certain adult cells, it constitutes a self antigen. Vaccines based on self antigens usually generate only a very modest and self-limited response, if they generate any response at all. But the frequency of cytotoxic T cells reactive against TERT observed in this study is comparable to the frequency typically observed for vaccines targeting powerful foreign antigen systems, such as the purified protein derivative (PPD) of tuberculosis. Cytotoxic T cell responses of this magnitude are sufficiently high to clear a pathological foreign agent from the affected host.

The high frequency of TERT-specific cytotoxic T cells generated in response to the TERT dendritic cell vaccine was consistent throughout the trial. The design of the trial required that the patients all be treated within a 5-week period. In the normal course of commercial use, further immunizations would be given periodically, maintaining or increasing the high level of TERT specific T cells for as long as desired.

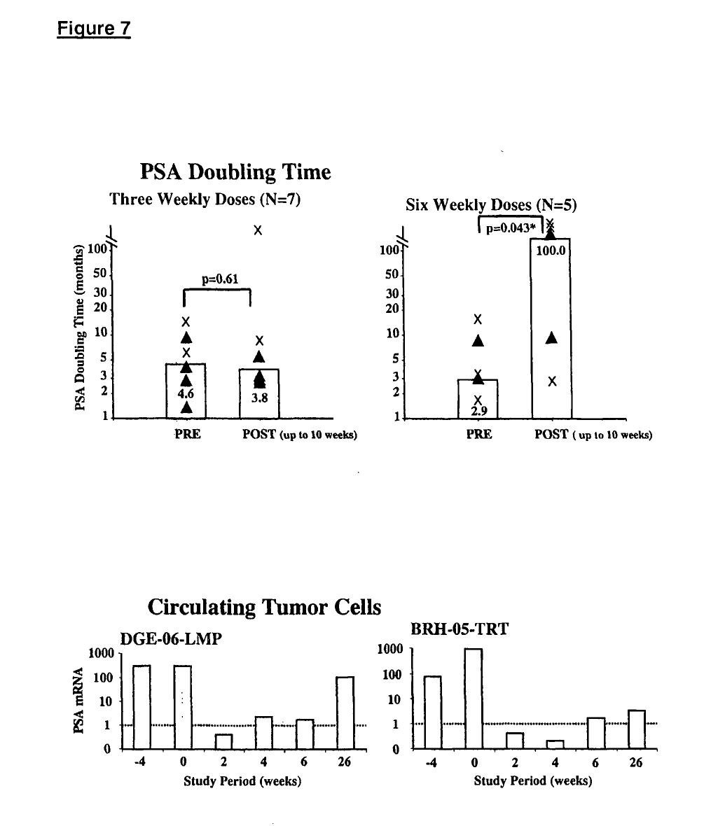

FIG. 7 shows the clinical status of patients who were treated. Circulating levels of prostate specific antigen (PSA), which correlates with active prostate cancer, was measured on an ongoing basis. The level of PSA increased with a doubling time of several days before therapy. As shown in the Upper Panel, patients that were vaccinated three times with the dendritic cell vaccine continued to show increasing PSA levels. However, all but two of the patients vaccinated six times showed no further increase in PSA levels for the 10 weeks of the study.

The Lower Panel shows the level of circulating tumor cells expressing PSA, measured by real-time PCR amplification of mRNA extracted from peripheral blood cells.

The level of circulating tumor cells measured in these patients prior to immunization was 100- to 1000-fold higher than what is seen in men without prostate cancer (indicated by the horizontal line). Circulating tumor cells became undetectable in this patient after the initial vaccination, and remained undetectable for 3 months after the final vaccination. The majority of other patients in the study showed a similar clearance of tumor cells from the circulation following treatment with the TERT dendritic cell vaccine.

Data from this trial show that the TERT dendritic cell vaccine generates a potent TERT-specific cytotoxic T cell response, which in turn mediates clearance of circulating cancer cells from the treated patients.

Adaptations of the invention can be made as a matter of routine optimization, without departing from the scope of the following claims

The invention claimed is:

1. A dendritic cell vaccine for treating cancer, comprising dendritic cells differentiated from hES cells in a pharmaceutical excipient, wherein the genome of the dendritic cells has been recombinantly altered so that the cells express one or more immunogenic epitopes of TERT.

2. The vaccine of claim 1, wherein the dendritic cells express a protein comprising at least 1000 consecutive amino acids of human TERT.

3. The vaccine of claim 1, wherein the dendritic cells express a protein comprising at least 1000 consecutive amino acids of human TERT having one or more amino acid changes from the natural human TERT sequence that result in the protein being devoid of telomerase catalytic activity in the presence of telomerase RNA component.

4. The vaccine of claim 1, wherein the dendritic cells express a plurality of protein fragments which between them include at least 1000 consecutive amino acids of human TERT.

5. The vaccine of claim 1, wherein the dendritic cells are genetically altered such that said immunogenic TERT epitopes are expressed under control of a promoter that is inducible by combining the cells with an inducing compound.

6. The vaccine of claim 1, wherein the dendritic cells are homozygous at the HLA-A locus.

7. A population of human dendritic cells differentiated from hES cells, said dendritic cells having a genome that has been recombinantly altered so that the cells express a tumor specific antigen under control of a promoter that is inducible by combining the cells with an inducing compound.

8. The dendritic cells of claim 7 wherein the tumor specific antigen comprises one or more immunogenic epitopes of TERT.

9. The dendritic cells of claim 7, wherein the inducing compound is tetracycline, isopropyl-β-D-thiogalactopyranoside, picolinic acid or desferrioxamine.

10. A method of treating cancer in a subject, comprising administering to the subject a vaccine according to claim 1.

11. The method of claim 10, comprising identifying one or more HLA allotype(s) of the subject, and treating the subject with dendritic cells having HLA allotype(s) that match those of the subject.

12. A cell combination for manufacturing a cellular vaccine for treating cancer, comprising:
a) dendritic cells characterized by all three of the following criteria:
i) they have been differentiated from human embryonic stem (hES) cells,
ii) they either express CD86, HLA Class II, and either or both of CD80 and CD83;
or they express Dec 205 and F4/80 or IL-12, but not CD80 or CD86;
iii) the genome of the cells has been recombinantly altered so that the cells express a protein comprising one or more immunogenic epitopes of telomerase reverse transcriptase (TERT); and
b) the hES cell line from which the dendritic cells were derived.

13. A combination of pharmaceutical preparations for treating cancer, comprising:
a) a first preparation comprising dendritic cells, characterized in that they have been derived from an hES cell line, express Dec 205 and either F4/80 or IL-12, but not CD80 or CD86; and have a genome that has been recombinantly altered so that the cells express a protein comprising one or more immunogenic epitopes of telomerase reverse transcriptase (TERT); and
b) a second preparation comprising an adjuvant, which upon administration preceding or simultaneous to administration of the dendritic cells at or near the same site causes the dendritic cells to increase expression of HLA Class II or to increase migration in vivo.

14. The combination of claim 13, wherein the adjuvant is selected from imiquimod, and polyarginine.

15. A method of treating cancer in a subject, comprising administering to the subject a combination of pharmaceutical preparations or vaccines according to claim 13.

16. A combination of separate vaccines for treating cancer, comprising:
a) a first vaccine according to claim 1; and
b) a non-cellular second vaccine comprising said immunogenic epitopes of TERT in the form of a protein or peptide, or a nucleic acid encoding said TERT epitopes.

17. The combination of claim 16, wherein the second vaccine comprises an adenovirus expression vector encoding said TERT epitopes.

18. A method of treating cancer in a subject using the combination of claim 16, comprising administering the first vaccine to the subject so as to prime an immunological response to human TERT; and then administering the second vaccine to the subject on one or more occasions so as to boost the anti-TERT response in the subject.

19. A method for activating dendritic cells according to claim 7, comprising combining said dendritic cells with said inducing compound.

20. A method for treating cancer in a subject, comprising administering dendritic cells according to claim 7 to the subject after they have been activated with the inducing compound.

21. The vaccine of claim 1, wherein the genome of the cells was recombinantly altered in the manner stated while the cells were still hES cells.

22. The vaccine of claim 1, wherein the genome of the cells was recombinantly altered in the manner stated while the cells were proliferative hematopoietic or dendritic cell precursors.

23. The vaccine of claim 1, wherein the genome of the cells was recombinantly altered in the manner stated using a DNA plasmid, a lentiviral vector, or retroviral vector.

24. The vaccine of claim 1, wherein the genome of the cells was recombinantly altered in the manner stated by homologous recombination.
